(19) United States
(12) Patent Application Publication
(10) Pub. No.: US 2025/0010039 A1
(43) Pub. Date: Jan. 9, 2025

(54) MEDICAL TUBE AND CABLE ORGANIZER

(71) Applicant: HH Medical, LLC, Foristell, MO (US)

(72) Inventor: Jason R. HAUK, Foristell, MO (US)

(73) Assignee: HH Medical, LLC, Foristell, MO (US)

(21) Appl. No.: 18/763,412

(22) Filed: Jul. 3, 2024

Related U.S. Application Data
(60) Provisional application No. 63/525,318, filed on Jul. 6, 2023.

(51) Int. Cl. A61M 25/02; F16L 3/223

(52) U.S. Cl. CPC A61M 25/02; F16L 3/223; A61M 2025/028; A61M 2205/583

(57) ABSTRACT

A foam organizer for tubing, cables, cords, and the like. The organizer comprises a substantially planar plate having a plurality of keyhole-shaped slots formed therein that extend from an edge of the plate toward a central portion of the plate. The plate also has a designated area associated with each slot for receiving a label.

### CROSS-REFERENCE TO RELATED APPLICATION

This application claim priority to U.S. Provisional Patent Application No. 63/525,318, filed Jul. 6, 2023, the entire disclosure of which is incorporated herein by reference.

### BACKGROUND

Health care providers can use a multitude of intravenous (IV) tubing and medical devices with a single patient resulting in a convoluted mess of tubing and cables. In addition, the relatively large number of medical devices crowds the amount of patient space and treatment area around a patient. The medical devices generally each require a power cable and some form of cord running to the patient for monitoring purposes. With the addition of the IV tubing that may be required. it is easy to envision a tangled nest of cables and tubes making it difficult for the patient to move about the room and for health care providers to render treatment to the patient.

Conventional cord organization devices are typically designed to specifically clamp onto specific surfaces in a patient's room. Although such devices may be useful, they tend to be bulky and difficult to secure and remove. In addition, such devices are often limited to the specific types of surfaces or shapes to which they may be applied.

In the medical industry, liability for mistakes is a significant concern due to the huge costs that have been associated with such mistakes for many years. Among those mistakes that may be made, IV administration of fluids and/or pharmaceuticals present a particular liability. IV administration of a fluid and/or pharmaceutical includes direct injections or infusions of the fluid and/or pharmaceutical into the blood stream of a patient through a vein through an IV tube. Convenient labeling of the tubes is needed to reduce risk of misadministration.

Another concern is the spread of germs and bacteria in a hospital or clinic setting. Hospitals and clinics are becoming increasingly aware that their environments play an important role in the transmission of infections and it is becoming clear that the fixtures, curtains, and devices are sites of potential contamination. Consequently, there is a need for a cord and tube organizer that not only provides a user with the ability to quickly secure it to a number of different shapes and surfaces but also can be easily removed for disinfection or disposal thus reducing potential contamination.

Organizers have been proposed to help organize the tangle of tubing, cables, and the like but such organizers can be overly expensive, which drives up the cost of health care, as well as cumbersome and limited to specific devices.

### SUMMARY

Aspects of the present disclosure facilitate organization of tubing, cables, and the like in a medical or healthcare setting.

In an aspect, an organizer comprises a substantially planar plate having a plurality of keyhole-shaped slots formed therein. The slots extending from an edge of the plate toward a central portion of the plate and each is configured for receiving a tube or cable.

Other objects and features of the present invention will be in part apparent and in part pointed out herein.

### BRIEF DESCRIPTION OF THE DRAWINGS

FIG. 1 is a top perspective view of an organizer according to an embodiment.

FIG. 2 is a top view of the organizer of FIG. 1 according to an embodiment.

FIG. 3 is an enlarged view of a slot formed in the organizer of FIG. 1 for receiving a tube or cable according to an embodiment.

FIG. 4 illustrates the organizer of FIG. 1 having tubes and/or cables secured therein according to an embodiment.

FIG. 5 further illustrates a plurality of organizers of FIG. 1 having tubes and/or cables secured therein according to an embodiment.

Corresponding reference numbers indicate corresponding parts throughout the drawings.

### DETAILED DESCRIPTION

The features and other details of the concepts, systems, and techniques sought to be protected herein will now be more particularly described. It will be understood that any specific embodiments described herein are shown by way of illustration and not as limitations of the disclosure and the concepts described herein. Features of the subject matter described herein can be employed in various embodiments without departing from the scope of the concepts sought to be protected.

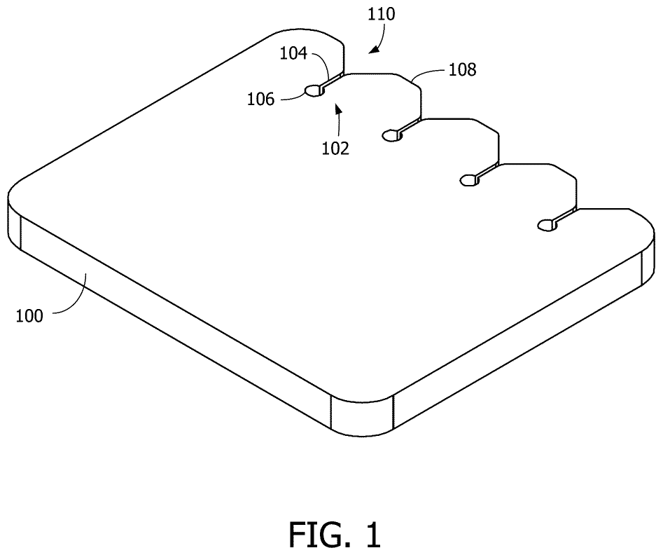

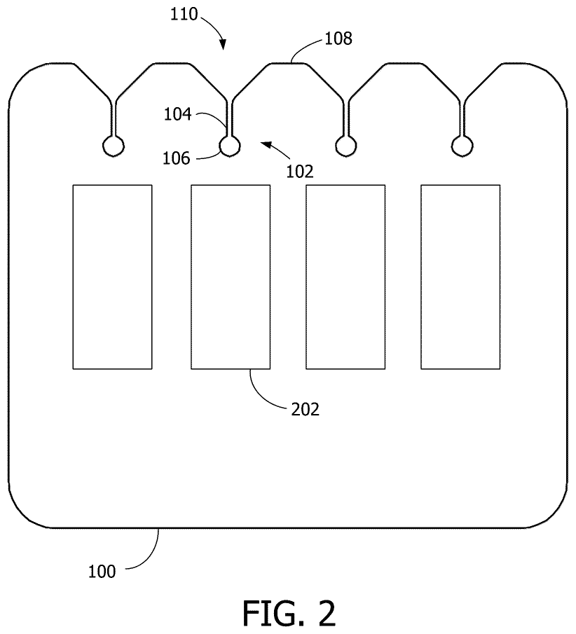

Referring to FIGS. 1 and 2, one example of an organizer 100 embodying aspects of the present disclosure is shown. In the illustrated embodiment, the organizer 100 is made from a lightweight, durable, and flexible closed-cell copolymer foam, such as ethylene-vinyl acetate (EVA). The organizer 100 is configured to selectively receive and retain IV tubing, cables, and the like. The organizer 100 comprises a substantially planar plate having a plurality of keyhole-shaped slots 102 formed therein. Each keyhole-shaped slot 102 forms a narrow opening 104 in the edge margin of the plate and terminate with a wider section 106. The slots 102 are shaped and sized to accommodate typical conduits, such as IV tubes, power cords, communication cables, oxygen tubes, feeding tubes, and the like, which are used in medical and healthcare settings. The plate material of organizer 100 is deformable such that the narrow opening 104 deforms to permit entry of the tube or cable and then is resilient enough to return to its narrow configuration when the tube or cable reaches the wider section 106. In this manner, tubes or cables may be secured within slots 102.

The organizer 100 embodying aspects of the present disclosure has a plurality of fingers 108 formed therein. The fingers 108 formed in the edge margin of organizer 100 are separated from each other and define slots 102. In the illustrated embodiment, fingers 108 are formed to define an opening 110 therebetween that is substantially wider than narrow slot 104 and configured such that a user can more easily insert a tube or cable into the slot 102.

In an embodiment, organizer 100 is generally rectangular and approximately 2.5 inches by 3.0 inches, and approximately 0.24 inches thick. In this embodiment, the wider section 106 of each slot 102 is generally circular and approximately 0.11 inches in diameter. It is to be understood that the size of organizer 100 may be adjusted to accommodate installation in different settings and to receive and secure different sizes of conduits (e.g., tubes and/or cables).

TABLE I
below, lists suitable characteristics of the material from
which organizer 100 is formed according to an embodiment:

| Physical Properties | Value | |
|---|---|---|
| Density-Nominal | 6 | lb/ft³ |
| Tensile Strength | 125 | psi |
| Tear Strength | 33 | lb/in |
| Elongation | 250% | |
| Asker Hardness | 60 | Asker C |
| Compression Set (50% 24 hr) | <10% | |
| Compression Strength (25%) | 32 | psi |
| Compression Strength (50%) | 50 | psi |
| Working Temperature Range | −60 to 224° | F. |
| Water Absorption (7 days) | 0.001 | lb/ft² |

| | |
|---|---|
| Flammability | Pass at 0.25″ |

It is to be understood that the values for the physical properties listed above are exemplary and a range of values (e.g., approximately +/−5%) would be suitable. XP-60 2-Stage Crosslinked Polyethylene is an example of a suitable material from which organizer 100 is formed.

Advantageously, organizer 100 includes a designated area 202 as shown in FIG. 2 according to one or more embodiments. In the example of FIG. 2, the designated area 202 comprises a rectangle associated with each slot 102 for receiving a handwritten or printed label displaying information concerning what is received in the associated slot 102. The organizer 100 can be disposed or disinfected after each patient use.

Although organizer 100 is illustrated as capable of accommodating four tubes and/or cables, it is to be understood that organizer 100 can be sized and shaped for fewer or more tubes and/or cables. In addition, it contemplated that organizer 100 may be formed in a larger sheet having sections separated by perforations such that the sections can be easily split into individual organizers 100 having the same or different number of slots 102 depending on the installation.

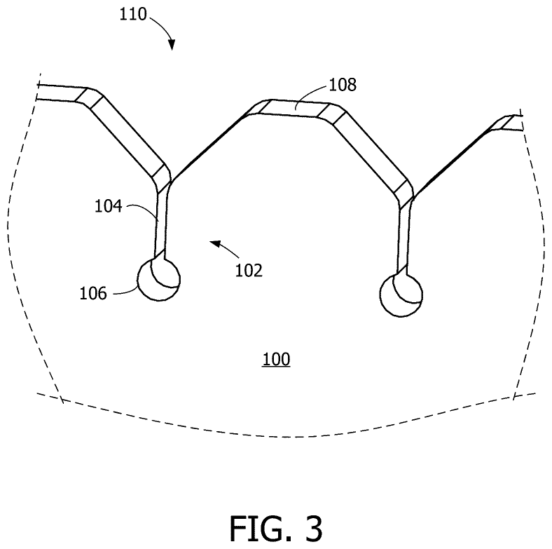

FIG. 3 is an enlarged, partial view of organizer 100 illustrating an exemplary slot 102 formed in the organizer for receiving a tube or cable according to an embodiment.

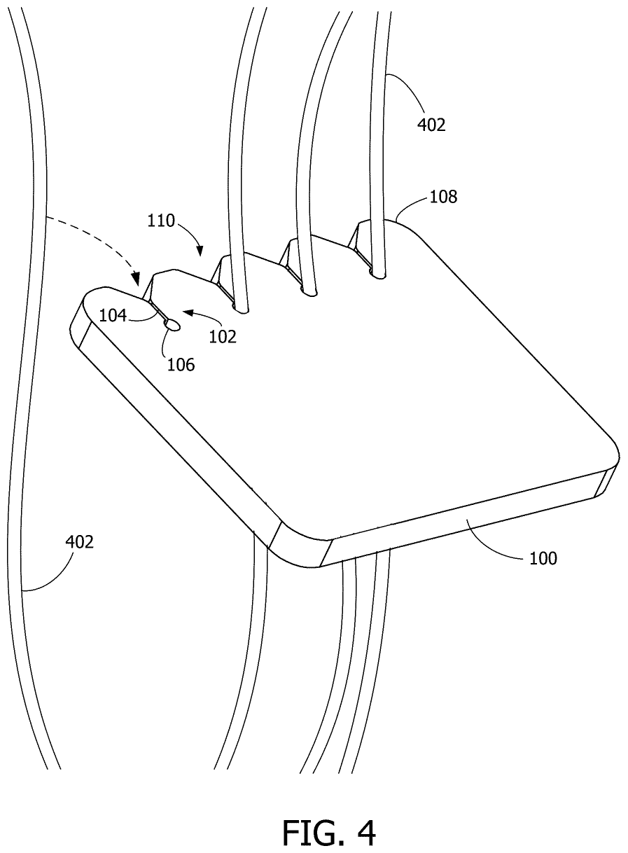

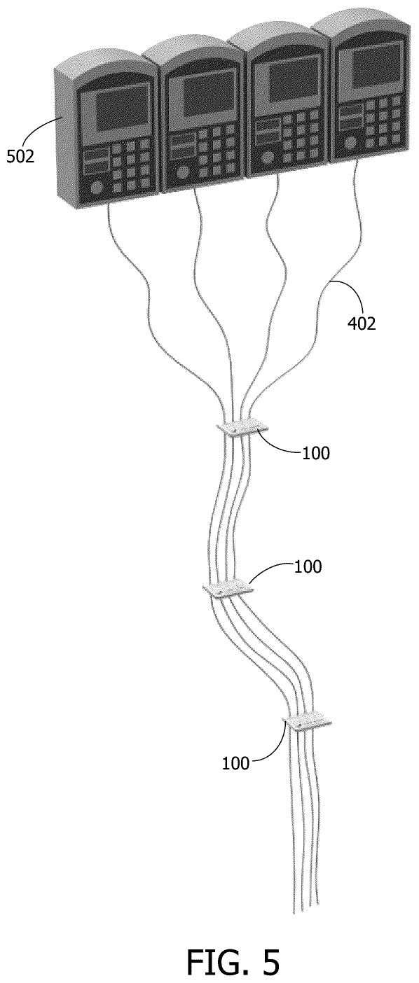

FIGS. 4 and 5 illustrate organizer 100 having example tubes and/or cables 402 removably secured therein according to one or more embodiments. It is to be understood that tubes and/or cables 402 may be typical IV tubes, power cords, communication cables, or the like used in medical and healthcare settings. As shown in the example installation of FIG. 5, tubes and/or cables 402 are coupled to a plurality of medical devices 502.

When introducing elements of the invention or embodiments thereof, the articles “a,” “an,” “the,” and “said” are intended to mean that there are one or more of the elements. The terms “comprising,” “including,” and “having” are intended to be inclusive and mean that there may be additional elements other than the listed elements.

Not all of the depicted components illustrated or described may be required. In addition, some implementations and embodiments may include additional components. Variations in the arrangement and type of the components may be made without departing from the spirit or scope of the claims as set forth herein. Additional, different or fewer components may be provided and components may be combined. Alternatively, or in addition, a component may be implemented by several components.

The above description illustrates embodiments by way of example and not by way of limitation. This description enables one skilled in the art to make and use aspects of the invention, and describes several embodiments, adaptations, variations, alternatives and uses of the aspects of the invention, including what is presently believed to be the best mode of carrying out the aspects of the invention. Additionally, it is to be understood that the aspects of the invention are not limited in its application to the details of construction and the arrangement of components set forth in the following description or illustrated in the drawings. The aspects of the invention are capable of other embodiments and of being practiced or carried out in various ways. Also, it will be understood that the phraseology and terminology used herein is for the purpose of description and should not be regarded as limiting.

It will be apparent that modifications and variations are possible without departing from the scope of the invention defined in the appended claims. As various changes could be made in the above constructions and methods without departing from the scope of the invention, it is intended that all matter contained in the above description and shown in the accompanying drawings shall be interpreted as illustrative and not in a limiting sense.

In view of the above, it will be seen that several advantages of the aspects of the invention are achieved and other advantageous results attained.

The Abstract and Summary are provided to help the reader quickly ascertain the nature of the technical disclosure. They are submitted with the understanding that they will not be used to interpret or limit the scope or meaning of the claims. The Summary is provided to introduce a selection of concepts in simplified form that are further described in the Detailed Description. The Summary is not intended to identify key features or essential features of the claimed subject matter, nor is it intended to be used as an aid in determining the claimed subject matter.

1. An organizer comprising a substantially planar plate having a plurality of keyhole-shaped slots formed therein along at least one edge margin of the plate, the slots extending from an edge of the plate toward a central portion of the plate.

2. The organizer of claim 1, wherein each of the slots is shaped and sized to receive and retain a conduit.

3. The organizer of claim 2, wherein the conduit comprises one or more of an IV tube, a power cord, a cable, an oxygen tube, and a feeding tube.

4. The organizer of claim 2, wherein the plate comprises a designated area associated with each of the slots for labeling the conduit received within the associated slot.

5. The organizer of claim 1, wherein each of the keyhole-shaped slots comprises a narrow slot section extending from the edge of the plate and terminating at a wide slot section.

6. The organizer of claim 5, wherein the wide slot section of each of the slots is substantially circular in shape and has a diameter between about 0.15 inches and about 0.07 inches.

7. The organizer of claim 5, wherein each of the slots defines a pair of fingers extending from the edge margin of the plate on either side of the slot between the pair of fingers, each pair of fingers defining an opening therebetween that is substantially wider than the narrow slot section of the slot therebetween, the opening configured to facilitate insertion of a conduit into the narrow slot section between the pair of fingers.

8. The organizer of claim 1, wherein each of the slots has a length of at least about 0.3 inches.

9. The organizer of claim 1, wherein the plate has a thickness between about 0.2 inches and 0.4 inches.

10. The organizer of claim 1, wherein the plate comprises EVA foam.

11. The organizer of claim 1, wherein the plate is disposable.
